(19) United States
(12) Patent Application Publication
(10) Pub. No.: US 2026/0120512 A1
(43) Pub. Date: Apr. 30, 2026

(54) AUTOMATED SYSTEM AND METHOD FOR GENERATING SIMILARITY METRICS IN MULTIMODAL DATA STREAMS

(71) Applicant: Mirrim LLC, Fort Worth, TX (US)

(72) Inventor: Jennifer Galanis, Fort Worth, TX (US)

(21) Appl. No.: 19/369,938

(22) Filed: Oct. 27, 2025

Related U.S. Application Data
(60) Provisional application No. 63/712,056, filed on Oct. 25, 2024.

(51) Int. Cl. G06V 40/20; A61B 5/16; G06F 18/241; G06V 10/26; G06V 10/30; G06V 10/74; G06V 10/764; G06V 10/82; G06V 10/94; G06V 40/10; G06V 40/50

(52) U.S. Cl. CPC G06V 40/20; A61B 5/165; G06F 18/241; G06V 10/26; G06V 10/30; G06V 10/761; G06V 10/764; G06V 10/82; G06V 10/945; G06V 40/10; G06V 40/50

(57) ABSTRACT

A system and method of quantifying and evaluating interpersonal communications. Audio and physical gestures/movement output of subjects of an interaction are analyzed to determine the quality of the interaction. Features of the interaction may be classified and compared to predefined signals, and similarity metrics in behavior between different subjects are measured to assess level of rapport. A rapport rating is generated based on the degree of correlation and confirmed by measuring stress levels of each subject.

### CROSS REFERENCE TO RELATED APPLICATIONS

This application claims priority to U.S. Provisional Patent Application No. 63/712,056, filed Oct. 25, 2024, which is hereby incorporated by reference in its entirety.

### FIELD OF THE INVENTION

Embodiments of the present invention generally relate to automated systems and methods for quantifying and evaluating interpersonal communications between two or more humans based on computer processing of multimodal data streams (e.g., audio and visual). More specifically, preferred embodiments of the present invention relate to determining the quality of an interaction via extracted multimodal data metrics and providing performance feedback to each subject of one or more interactions.

### BACKGROUND

Globalization along with the rapid growth of artificial intelligence are causing a paradigm shift in today's workforce. In order to be competitive, companies need to be more dynamic, interconnected and flexible; therefore, executives are prioritizing soft skills in their employees.

Soft skills include interpersonal communication, listening, and empathy. They are not only essential for the external face of the company (interacting with customers), but also for the internal efficiency of operations (leadership and team management, a supportive work environment, and employee retention).

In reality, however, a large soft skills gap exists in the workforce. According to the Society for Human Resource Management, one in five Americans leave their current job due to bad company culture, costing businesses an estimated $44 billion dollar loss annually. After 18 months at work, approximately 40% of new hires show problems with soft skills and are subsequently terminated, or leave under pressure, or receive disciplinary action or significantly negative performance reviews.

Unlike technical “hard” skills, soft skills are challenging to teach and assess. They require active interactions with others on an ongoing basis, and people must demonstrate a willingness to acknowledge and accept behavioral feedback. Additionally, the environment as well as other people and cultural norms impact the development and use of an individual's soft skills. For these reasons, soft skill learning and assessments do not depend solely on a single person, but rather a person interacting with other people in a particular environment. Soft skills also require lifelong learning and practice.

Since soft skills are rooted in emotion and relationships, the system and methods presented here use subconscious behavioral displays people make in order to assess the level of connection or rapport between two or more people. People who feel more connected to each other are more likely to copy, mimic, mirror, or imitate each other's behaviors. This unconscious mimicry can be broad in scope, and includes facial expressions, body gestures and postures, and speech patterns.

There are many approaches used to try to assess people's interpersonal communication skills. Here the methods are roughly divided by the timing of the assessment in relation to when the interpersonal communication event or events occur-before, during, or after.

Assessments that occur after interactions include 360-degree feedback, downward feedback (supervisor rates a subordinate), upward feedback (subordinate rates a supervisor). These are performed after a significant time periods have passed, resulting in problems with remembering events. Evaluations can be riddled with biases (ex. primacy effect, halo effect, regency and spillover effects, and central tendency biases). Subordinates may not share critiques or give negative feedback due to a fear of risking their employment status. Since these evaluations are time and labor intensive and depend on the subjective skills of the human grader, these assessments are typically performed on an annual bases, if at all.

Assessments during an interaction include teaching/coaching sessions, and behavioral or traditional interviews, and can be applied to the hiring process and/or job training. Ideally, a communication expert observes, grades, and provides feedback on a person's interpersonal skills as they interact with other people in the real world or in simulated environments. These sessions are time and labor intensive, which limits their widespread use. Often the rater is an active participant in the conversation, which can lead to distraction and missing subtle cues indicative of bad communication styles.

Assessments before an interaction are typically used during the hiring process in an attempt to predict a person's communication style and filter potential candidates. These include personality tests, psychometric exams, and AI evaluations. Despite their widespread use, these methods have opaque assessment criteria, introduce biases, and do not accurately predict performance during real world conversations.

### BRIEF DESCRIPTION OF THE DRAWINGS

FIG. 1 illustrates a schematic overview of a computing device, in accordance with an embodiment of the present invention;

FIG. 2 illustrates a network schematic of a system, in accordance with an embodiment of the present invention;

FIG. 3 illustrates a schematic overview of a system for quantifying and evaluating interpersonal interactions, in accordance with an embodiment of the present invention;

FIG. 4 illustrates a schematic overview of an exemplary user interface, in accordance with an embodiment of the present invention; and

FIG. 5 illustrates a schematic overview of an exemplary method of quantifying and evaluating interpersonal interactions, in accordance with an embodiment of the present invention.

### SUMMARY OF THE INVENTION

According to an embodiment of the present invention, a computer-implemented method for segmenting and classifying multimodal data based on feature extraction and calculated distance metrics of the behavior of two or more individuals depicted in the multimodal data, the method executed by one or more processors and comprises: (a) capturing a first set of multimodal data, said first set of multimodal data comprising (i) audio data, wherein the audio data includes vocal acoustic and linguistic data and (ii) imaging data, wherein the imaging data includes data feature data; (b) segmenting the first set of multimodal data into a first subject multimodal dataset and a second subject multimodal data set; (c) registering, for each of the first subject multimodal dataset and second subject multimodal data set: (i) individual-specific coordinate frames, and (ii) denoising using a trained filter; (d) generating, data feature outputs for a first individual in the first subject multimodal dataset, based at least in part on the individual-specific coordinate frames registered in the first subject multimodal dataset; (e) generating, data feature outputs for a second individual in the second subject multimodal dataset, based at least in part on the individual-specific coordinate frames registered in the second subject multimodal dataset; (f) analyze data feature outputs of the first individual and the second individual to generate a coordinated data feature output dataset; (g) calculating similarity metrics in the coordinated data feature output dataset to assess common behavioral characteristics between first individual and second individual; (h) aggregating the similarity metrics to generate a score or grade for the quality of the interaction, or a rapport estimate; (i) identifying problematic behaviors in said similarity metrics in the coordinated data feature output dataset and generating a problematic behavior dataset; (j) linking events in said problematic behavior dataset to time points in multimodal data; and (k) outputting the problematic behavior dataset as linked to the multimodal data to a graphical user interface.

According to an embodiment of the present invention, imaging data comprises at least one imaging modality selected from optical imaging, digital imaging, analog imaging, ultrasound, IR imaging, and LiDAR imaging.

According to an embodiment of the present invention, capturing of the multimodal data is conducted via one or more sensors selected from the group comprising a motion sensor, an IR sensor, an ultrasound sensor, a digital imaging sensor, an analog imaging sensor, an audio sensor, a microphone, a physiological sensor, a vital sensor, an implantable sensor, a wearable sensor, and a LiDAR sensor.

According to an embodiment of the present invention, the multimodal data further comprises stress data.

According to an embodiment of the present invention, the stress data comprises bioinformatic information related to the first and second individual, wherein the bioinformatic information comprises one or more of: i) heart rate data; ii) respiration data; iii) perspiration data; iv) pupil dilation data; and v) micromovement data.

According to an embodiment of the present invention, the method further comprises the steps of: analyzing the stress data for the first individual to create a first individual stress level dataset; analyzing the stress data for the second individual to create a second individual stress level dataset; analyzing the problematic behavior dataset, the first individual stress level dataset and second individual stress level dataset to generate a rapport rating; and outputting, via said graphical user interface, standardized feedback related to said rapport rating.

According to an embodiment of the present invention, calculating similarity metrics and identifying problematic behaviors in said similarity metrics comprises applying a convolutional neural network-based segmentation model.

According to an embodiment of the present invention, convolutional neural network-based segmentation model is trained on an annotated behavioral analysis structure.

According to an embodiment of the present invention, a non-transitory computer readable medium storing instructions that, when executed by a processor, cause the processor to perform a method for segmenting and classifying multimodal data based on feature extraction and calculated distance metrics of the behavior of two or more individuals depicted in the multimodal data, the method executed by one or more processors and comprises: (a) capturing a first set of multimodal data, said first set of multimodal data comprising (i) audio data and (ii) imaging data, wherein the imaging data includes movement data; (b) segmenting the first set of multimodal data into a first subject multimodal dataset and a second subject multimodal data set; (c) registering, for each of the first subject multimodal dataset and second subject multimodal data set: (i) individual-specific coordinate frames, and (ii) denoising using a trained filter; (d) generating, movement outputs for a first individual in the first subject multimodal dataset, based at least in part on the individual-specific coordinate frames registered in the first subject multimodal dataset; (e) generating, movement outputs for a second individual in the second subject multimodal dataset, based at least in part on the individual-specific coordinate frames registered in the second subject multimodal dataset; (f) analyze movement outputs of the first individual and the second individual to generate a coordinated movement output dataset; (g) calculating similarity metrics in the coordinated movement output dataset to assess common behavioral characteristics between first individual and second individual; (h) identifying problematic behaviors in said similarity metrics in the coordinated movement output dataset and generating a problematic behavior dataset; (i) linking events in said problematic behavior dataset to time points in multimodal data; and (j) outputting the problematic behavior dataset as linked to the multimodal data to a graphical user interface.

According to an embodiment of the present invention, the non-transitory computer readable medium of claim 13, further comprises the steps of: analyzing the stress data for the first individual to create a first individual stress level dataset; analyzing the stress data for the second individual to create a second individual stress level dataset; analyzing the problematic behavior dataset, the first individual stress level dataset and second individual stress level dataset to generate a rapport rating; and outputting, via said graphical user interface, standardized feedback related to said rapport rating.

According to an embodiment of the present invention, a system comprising: a processor, a memory storing instructions, and a display, wherein the processor is configured to execute the instructions to perform a method to segment and classify multimodal data based on feature extraction and calculated distance metrics of the behavior of two or more individuals depicted in the multimodal data, the method comprising: (a) capturing a first set of multimodal data, said first set of multimodal data comprising (i) audio data and (ii) imaging data, wherein the imaging data includes movement data; (b) segmenting the first set of multimodal data into a first subject multimodal dataset and a second subject multimodal data set; (c) registering, for each of the first subject multimodal dataset and second subject multimodal data set: (i) individual-specific coordinate frames, and (ii) denoising using a trained filter; (d) generating, movement outputs for a first individual in the first subject multimodal dataset, based at least in part on the individual-specific coordinate frames registered in the first subject multimodal dataset; (e) generating, movement outputs for a second individual in the second subject multimodal dataset, based at least in part on the individual-specific coordinate frames registered in the second subject multimodal dataset; (f) analyze movement outputs of the first individual and the second individual to generate a coordinated movement output dataset; (g) calculating similarity metrics in the coordinated movement output dataset to assess common behavioral characteristics between first individual and second individual; (h) identifying problematic behaviors in said similarity metrics in the coordinated movement output dataset and generating a problematic behavior dataset; (i) linking events in said problematic behavior dataset to time points in multimodal data; and (j) outputting the problematic behavior dataset as linked to the multimodal data to a graphical user interface.

### DETAILED SPECIFICATION

Interpersonal communication is an interaction that is dynamically co-created by two or more people and is modulated by the circumstances of those people and the environment they are in. The instant system and method allow for increased accuracy in quantifying interpersonal communication by: simultaneously measuring two or more people interacting; in their usual environment (ex. at work); and/or in an unobtrusive manner (ex. does not require people to wear devices).

Assessments made by humans can suffer from biases (gender, race, ethnicity, religious views, socioeconomic status, dominance/hierarchy, etc.). According to an embodiment of the present invention, a system and method is detailed herein which overcomes biases from a rater based at least in part on: measuring subconscious biological markers and signals that are displayed in the course of communication; accounting for the totality of the interaction by using both voice and body motion signals; generating a similarity metric in behavior (rather than a predefined set of acceptable signals) in order to assess the level of rapport. Such predefined signals are usually cultural specific; simultaneously double-checking the rapport rating by measuring the stress level of each person individually; and providing standardizing feedback.

According to preferred embodiments of the present invention, the system detailed herein provides assessments that are easily scalable by: analyzing any multimodal data (e.g., audio, visual, biometric, optical, IR, ultrasonic, LiDAR, audio-visual data of interpersonal interactions); providing real-time analysis; and providing an automated system that rates the quality of the interactions and does not require an expert to use which allows users to catch problems earlier (when conflicts are easier to repair) and monitor trends in behavior. One of ordinary skill in the art would appreciate that there are numerous types of multimodal data that could be used with embodiments of the present invention, and embodiments of the present invention are contemplated for use with any appropriate type of multimodal data.

Embodiments of the present invention are directed towards monitoring and evaluating social interactions in relationships in situations which require teamwork, trust, empathy and/or diplomacy. For example, one embodiment may be used in business settings such as Human Resource Departments to monitor toxic work culture between employees, evaluate how team members work on a project, assess business meetings and conferences, including video conferencing. Another embodiment may be used in the healthcare industries to monitor the relationship between patients and providers, and the working relationship between providers (doctors, nurses, psychologists, social workers etc.). While another embodiment may be used by law enforcement, military personnel, and security to evaluate the bonds between team members and assess sensitivity and diplomacy when interacting with members of the community.

Embodiments of the present invention may further be used as part of training, education, and continuing education programs which can include managerial training and sales training, especially in careers where empathy in human interactions is critical in job performance. The training may be performed by academic institutions, independent training centers, government or internally within businesses. For example, one embodiment may be used by trainers, educators, and faculty to grade student performance and track student progress in time. In certain embodiments, the system and methods may be applied to Objective Structured Clinical Examinations or similar pre-licensing or licensing exams which are used by healthcare degree granting institutions or licensing bodies that evaluate the performance of medical doctors, doctor of osteopathy, nurses, physician assistants, psychology, other similar professionals, or any combination thereof. Furthermore, these systems and methods may be used as a tool to quantify the effectiveness of a communication training program and teaching methods, thereby allowing educators to standardize and improve teaching methods.

Embodiments of the present invention may further be used in clinical settings as a tool for monitoring the effectiveness of therapy for medical conditions that are known to affect interpersonal communication (ex. Major Depressive Disorder, Social Anxiety Disorder).

Embodiments of the present invention allow for transparency in measurements by breaking down the analysis of an interaction into various channels of communication as specialized graphs, flagging specific events and problematic behaviors and compiling those into an interactive list with explanations and recommendations. The embodiments further link those events to time points in the audio-visual signal, thereby easily allowing the user to reevaluate those specific behaviors.

According to an embodiment of the present invention, the system may be configured to detect subconscious signals between people. Specifically, certain embodiments relate to a system and method that detect and measure subconscious signals of communication between two or more people, uses those measurements to rate the quality of interaction, and provides a breakdown of that analysis and recommendations to the user.

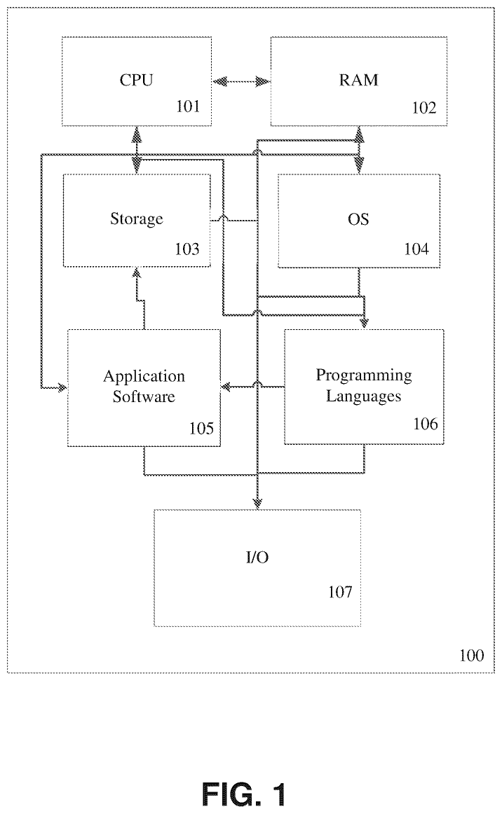

According to an embodiment of the present invention, the system and method is accomplished through the use of one or more computing devices. As shown in FIG. 1, One of ordinary skill in the art would appreciate that a computing device 100 appropriate for use with embodiments of the present application may generally be comprised of one or more of a Central processing Unit (CPU) 101, Random Access Memory (RAM) 102, and a storage medium (e.g., hard disk drive, solid state drive, flash memory) 103. Examples of computing devices usable with embodiments of the present invention include, but are not limited to, personal computers, smart phones, laptops, edge computing devices, mobile computing devices, tablet PCs and servers. The term computing device may also describe two or more computing devices communicatively linked in a manner as to distribute and share one or more resources, such as clustered computing devices and server banks/farms. One of ordinary skill in the art would understand that any number of computing devices could be used, and embodiments of the present invention are contemplated for use with any computing device. Additionally, the system may leverage one or more machine learning or artificial intelligence systems for computationally processing and modeling various multimodal datasets and elements, including, but not limited to, through the use of predictive models and other neural network models.

As used herein, the term “predictive model” refers to a computational model configured to process multimodal data, such as imaging-derived or audio-derived features, which may be utilized alongside or together with clinical data to estimate one or more quantitative indicators of behavioral conditions or patterns for individuals. The predictive model may be trained using labeled, unlabeled, semi-labeled, and/or historical datasets to learn relationships between features. The model may include, without limitation, regression models, ensemble methods, neural networks, or other machine-learning architectures capable of generating patient-specific predictions or scores based on multimodal input data. Unless otherwise specified, the term encompasses computational approaches that integrate biomechanical and clinical features to assess or predict the functional or behavioral state of one or more individuals. As used herein, the term “similarity metric” refers to a quantitative measure of distance between two signals, features or datasets. Metrics include, but are not limited to, distance L1, Euclidean distance L2, cosine similarity, Jaccard metrics, Hamming distance, correlations, entropy, divergences, edit distance, dynamic time warping, area-based measure, set-based measure, and adaptive similarity metric selection strategy or generalized forms of such methods. These metrics may be embedded into or filtered by machine learning process as defined in the methods of this document.

As used herein, the term “baseline” refers to a reference condition or comparative measure used to evaluate changes in the functional or predicted behavioral pattern(s). The baseline may be determined from one or more prior data points pulled from a multimodal dataset related to an individual, from population-level reference data matched by demographic or clinical factors. Or, in combination with or alternatively from a model-derived benchmark representing a normative physiological or behavioral state. Comparison of a computed behavioral score to the baseline enables assessment of an interaction or interaction set, or deviation from expected behavioral function(s). Unless otherwise specified, the term encompasses static and temporal reference values, including individual historical data and population benchmarks that provide contextual information for evaluating current and predicted individual-specific results.

As used herein, the term “temporal model” refers to a computational model configured to analyze or predict time-dependent changes in behavioral or clinical parameters for an individual. The temporal model may receive as input one or more previously computed indicators from a multimodal dataset, such as a baseline comparison, or other historical behavioral measurement, and generate a prediction representing how those parameters are expected to evolve over one or more future intervals/interactions. The model may capture temporal relationships, progression rates, or behavioral trends using statistical and/or machine learning architectures including, without limitation, regression models, autoregressive models, state-space models, hidden Markov models, recurrent neural networks, long short-term memory networks, and transformer-based sequence models. Unless otherwise specified, the term encompasses computational approaches that lean from sequential or longitudinal data to predict future states of a behavioral state or pattern, thereby enabling individual specific planning.

As used herein, the term “model-uncertainty metric” refers to a quantitative measure of confidence, reliability, or variability associated with a prediction generated by a computational model. The model uncertainty metric may reflect the degree of dispersion, variance, or entropy in predicted outputs and may be computed using techniques such as probabilistic inference, Bayesian estimation, dropout sampling, ensemble variance, or confidence calibration. In the context of patient-specific modeling, the uncertainty metric is used to determine when additional multimodal imaging or monitoring is warranted, such as when uncertainty regarding a predicted functional parameter exceeds a defined threshold. Unless otherwise specified, the term encompasses any statistical and/or machine learning-based measure that quantifies prediction confidence or expected error for outputs if a predictive model or temporal model used in planning.

In an exemplary embodiment according to the present invention, data may be provided to the system, stored by the system and provided by the system to users of the system across local area networks (LANs) (e.g., office networks, home networks) or wide area networks (WANs) (e.g., the Internet). In accordance with the previous embodiment, the system may be comprised of numerous servers communicatively connected across one or more LANs and/or WANs. One of ordinary skill in the art would appreciate that there are numerous manners in which the system could be configured and embodiments of the present invention are contemplated for use with any configuration.

In general, the system and methods provided herein may be consumed by a user of a computing device whether connected to a network or not. According to an embodiment of the present invention, some of the applications of the present invention may not be accessible when not connected to a network, however a user may be able to compose data offline that will be consumed by the system when the user is later connected to a network.

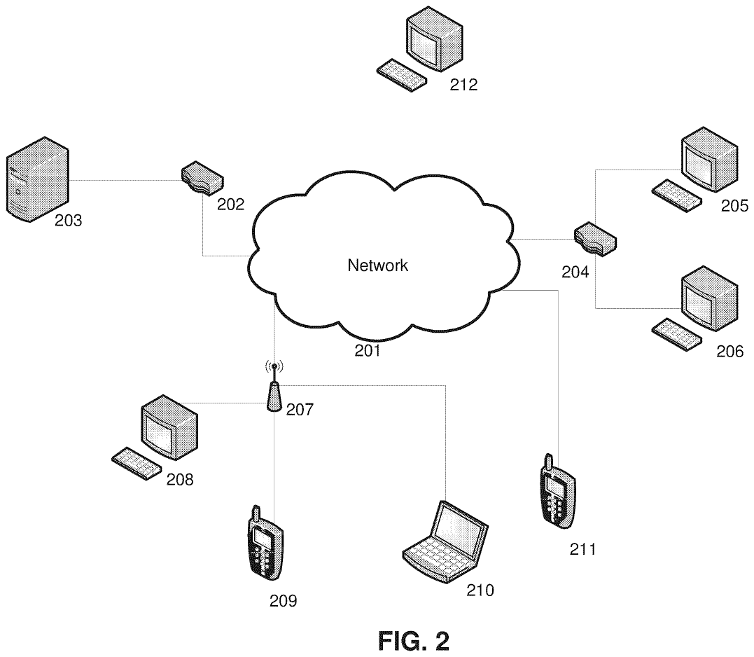

Referring to FIG. 2, a schematic overview of a system in accordance with an embodiment of the present invention is shown. The system is comprised of one or more application servers 203 for electronically storing information used by the system. Applications in the server 203 may retrieve and manipulate information in storage devices and exchange information through a WAN 201 (e.g., the Internet). Applications in server 203 may also be used to manipulate information stored remotely and process and analyze data stored remotely across a WAN 201 (e.g., the Internet).

According to an exemplary embodiment, as shown in FIG. 2, exchange of information through the WAN 201 or other network may occur through one or more high speed connections. In some cases, high speed connections may be over-the-air (OTA), passed through networked systems, directly connected to one or more WANs 201 or directed through one or more routers 202. Router(s) 202 are completely optional and other embodiments in accordance with the present invention may or may not utilize one or more routers 202. One of ordinary skill in the art would appreciate that there are numerous ways server 203 may connect to WAN 201 for the exchange of information, and embodiments of the present invention are contemplated for use with any method for connecting to networks for the purpose of exchanging information. Further, while this application refers to high speed connections, embodiments of the present invention may be utilized with connections of any speed.

Components of the system may connect to server 203 via WAN 201 or other network in numerous ways. For instance, a component may connect to the system i) through a computing device 212 directly connected to the WAN 201, ii) through a computing device 205, 206 connected to the WAN 201 through a routing device 204, iii) through a computing device 208, 209, 210 connected to a wireless access point 207 or iv) through a computing device 211 via a wireless connection (e.g., CDMA, GMS, 3G, 4G, 5G) to the WAN 201. One of ordinary skill in the art would appreciate that there are numerous ways that a component may connect to server 203 via WAN 201 or other network, and embodiments of the present invention are contemplated for use with any method for connecting to server 203 via WAN 201 or other network. Furthermore, server 203 could be comprised of a personal computing device, such as a smartphone, acting as a host for other computing devices to connect to.

Embodiments of the present invention provide a system and method that measures subconscious signals of communication between two or more people, uses those measurements to rate the quality of interaction, and provides a breakdown of that analysis and recommendations to the user. In order to improve accuracy in the measurements, the method incorporates factors that estimate both the level of rapport between people and stress of individuals.

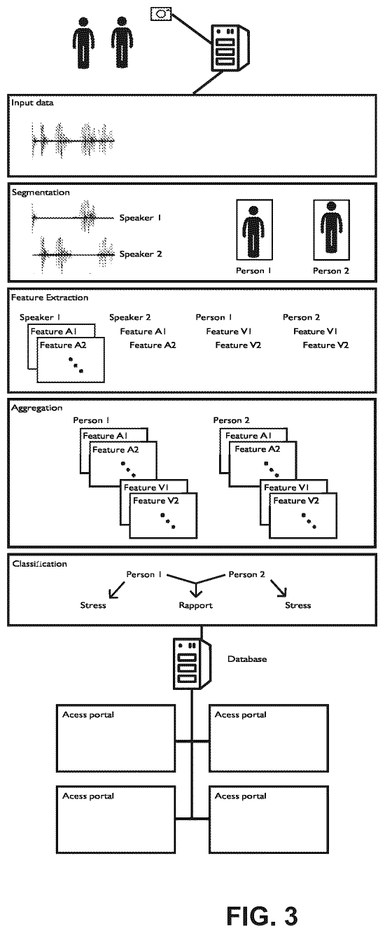

As shown in FIG. 3, the system of the present invention includes audio-visual equipment such as one or more video cameras, microphones, infrared (thermographic) cameras and/or sensors operably connected to a processing device such as a computer, laptop, smartphone, server, etc. For simplicity, the audio-visual equipment will be collectively referenced as camera. The camera captures video and audio (i.e., audio-visual input) from two or more subjects and segments the audio-visual input. The audio-visual input signal may be segmented according to subjects (e.g., people) in the image frame. For example, in a room where subjects 1, 2, and 3 are being observed, the audio-visual (AV) signal is segmented into AV1, AV2 and AV3. The AV signal may be segmented using any known method such as computer vision methods with object detection algorithm (i.e., a detector) trained to detect the full body, upper body, or face of a person in an image frame. The detector may be applied as needed. For example, object detection may be applied to the first frame, and again when the error estimating the accuracy of the tracker exceeds a threshold limit.

The purpose of this method is to: count the number of people in an image frame; draw a region of interest (ROI), a set of (x,y) coordinates in the image matrix that contains a person; track the ROI in time by updating the set of (x,y) coordinates in a series of image frames; and save the output for further processing (detailed below).

An audio signal from audio recorded of the subjects may similarly be segmented according to subject and may or may not be part of the AV signal discussed above. For example, the audio signal may be an audio signal from a standalone microphone or may be part of an AV signal captured by a camera with built-in microphone. The audio signal may be segmented according to subject using speaker diarization methods that can be obtained via standard coding libraries or through machine learning techniques such as recurrent neural networks (RNN), long sort term memory (LSTM) techniques, etc. The purpose of this method is to detect and count the number of people speaking, separate the audio signal into tracks/channels of homogeneous segments corresponding to each person, and save the output for further processing (detailed below).

According to an embodiment of the present invention, predefined features are extracted from the segmented multimodal dataset, which may include, but is not limited to, audio and visual signals. The set of features includes, but is not limited to, the following: features extracted from each ROI-cropped image frame used as input for an interaction evaluation algorithm configured to rate and determine the nature of the interaction (e.g., positive or negative) where the interaction may be classified on a continuum that ranges from extremely negative to extremely positive. The feature extraction process may: generalized motion detection-apply frame differencing to two consecutive image matrices or two image matrices separated by a static number (e.g., 5) of frames.

According to certain embodiments, body skeletonization/pose tracking may be utilized to provide a set of (x,y) coordinates that mark some or all of the following significant body points or nodes: neck, shoulder (L/R), elbow (L/R), wrist (L/R), hip (L/R), knee (L/R), ankle (L/R), heel (L/R), foot (L/R). These can be obtained via standard coding libraries or through computer vision machine learning techniques such as convolutional neural networks (CNN).

An affine transformation may be applied to the set of (x,y) coordinates so that the coordinates are rescaled with respect to an anchor reference point. For example, the neck may be defined as (0,0) or a standard unit length may be defined as the length of the face (1 L=distance from (x,y) of the chin to (x,y) of the top of the forehead), and all other (x,y) coordinates for body points are adjusted accordingly. An additional three-dimensional mapping transform may be applied, where the relative position of body coordinates in (x, y)-space are used to estimate the set of coordinates in (xt, yt, zt)-space.

Since nodes by themselves insufficiently capture the topology of body poses, associations between nodes may be used. For example, vectors (distance and angle) between pairs of connected nodes, vectors (distance and angles) between pairs of “significant” nodes (for example between two end nodes), distances between vectors and nodes, angles between vectors, planes created by 3 connected nodes, angles between planes and vectors, and angles between planes. Further derivative features may include but are not limited to velocities and accelerations of features as features change over time (subsequent frames in a video).

According to certain embodiments, hand landmark/detector/tracker(s) can be utilized to provide a set of (x,y) coordinates that mark some or all of the following significant points in the hand (L/R): thumb+4 fingers, marking 3 joints+1 tip per digit. These can be obtained via standard coding libraries or through computer vision machine learning techniques such as convolutional neural networks CNN. And a set of features is generated by methods analogous to those described in the pose tracking section may be used.

According to certain embodiments, facial landmark detector/tracker(s) can be utilized to provide mesh mapping of facial features for movement of the facial muscles, especially highlighting contours surrounding eyes, eyebrows, and mouth. These can be obtained via standard coding libraries or through computer vision machine learning techniques such as convolutional neural networks CNN. And a set of features is generated by methods analogous to those described in the pose tracking section may be used.

According to certain embodiments, gaze estimation systems can be utilized by tracking the iris within an eye. These can be obtained via standard coding libraries or through computer vision machine learning techniques.

According to certain embodiments, audio features extracted from each segmented audio channel may include: non-language based features, including: acoustic and prosodic elements; Fourier frequencies (including fundamental freq.); Mel-Frequency Cepstral Coefficients (MFCC); Energy and entropy of energy; Spectral domain (spectral centroid, spectral spread, spectral entropy, spectral flux, spectral rolloff, tilt); Chroma vector & standard deviation of chroma vector; Time duration of phonemes and words; Time duration of silence between phonemes and words.

According to certain embodiments, language-based features/conversational analysis systems can be utilized. From the diarization output described earlier, each output channel is processed to convert speech to text to vector format (text vectorization). Speech to text (STT) or automatic speech recognition (ASR) can be obtained via standard coding libraries. Text vectorization can occur via standard coding libraries that can include Bag of Words (BoW), weighted BoW (like term frequency-inverse document frequency TF-IDF, or BM25), embedded semantic and syntactic properties (like Word2Vec), transformers (like bidirectional encoder representations from vectorization BERT) or analogous methods.

Additional features may include: lexical repetition/echo utterance, whereby the system is configured to count the number of repeated words and phrases between speakers and/or count the use of jargon words and slang. In certain embodiments, turn taking is tracked, where duration of turns, frequency of taking turns and gaps and overlaps of dialogue are tracked.

According to an embodiment of the present invention, the audio and visual features of each subject may be aggregated where all features associated with each subject are combined according to subject. Machine learning can then be used in the following manner: 1) If multiple microphones and video cameras are present: triangulation techniques to link the location of the audio signal with the location of the person in the image; or 2) for one audio-visual source, the system may use features to link audio and visual signals. For example, an audio channel that contains voice at a given time t can be synchronized with movement of the lips (as extracted by facial landmark detection) at time t.

In the case of failure of these methods, a prompt for human user assistance will appear. The user will then match the appropriate audio and video channels. The user provided input will further train the matching algorithm. If the range of feature values are significantly different, for example greater than one order of magnitude, then the output may be dominated by the larger feature values. To correct for this error, normalization and/or standardization methods may be applied to some or all the features. For example, the features may be rescaled to have a maximum value of 1 and a minimum value of zero, may have values clipped, may be transformed via a log scaling or a z-score, or similar methods.

If segments of the original audio-video input are missing (for example by equipment failure) or feature data is missing (for example by objects moving out of the image frame), then the missing data may decrease the accuracy of the numerical analysis. Various methods for imputing missing values may be used, such as replacing the gap values with the value before the gap, the value after the gap, the mean value of the feature, applying a linear or spline interpolation, Kalman smoothing, machine learning techniques (such as k-nearest neighbor KNN, recurrent neural networks RNN, generative adversarial networks GAN, multilayer perceptrons MLP, etc.), or other similar methods.

The total number of features is the dimension of the analysis. For large feature sets, such as described in this document, there may be redundant and irrelevant features in the set. To reduce computational complexity, memory and time required for data processing, dimensionality reduction methods such as maximum relevance-minimum redundancy (MRMR), conditional mutual information maximization (CMIM), correlation coefficient, between-within ratio (BW-ratio), support vector machine-recursive feature elimination (SVM-REF), principal component analysis (PCA), etc. may be used.

In certain embodiments, if the audio-visual input is segmented into time-windows (1 minute or less) that overlap (max 75%), a set of features, as previously described, is extracted from each time-window. When comparing features or sets of features that describe different people, a similarity metric is used to calculate the distance between the features. Metrics include, but are not limited to, Euclidean distance, correlations, entropy, divergences, edit distance, dynamic time warping, area-based measure, set-based measure, and adaptive similarity metric selection strategy. When comparing features or sets of features that describe different people, these comparison of these features may include a time lag (+/−30 seconds maximum) with time-lag increments (0.5 seconds maximum).

According to an embodiment of the invention, a classification module or “classifier” is trained using labels which may be obtained via the following methods: 1) questioners from people in the interaction immediately after an interaction; 2) experienced/qualified human raters who observe the interaction; 3) bio-physiological markers (examples include heart rate, body temperature, hormones, etc.); or 4) for publicly posted audio-visual data (ex. YouTube), crowd-sourced ratings via an average score from posted comments.

As discussed, evaluating an interaction may include “stress” and “rapport” analysis. Stress measurements may entail analysis and classification of features associated with a single subject, or a group of subjects analyzed independently. Rapport measurements, on the other hand, generally entails analysis and classification of features associated with a group of subjects. A group of course includes two or more subjects. In addition, features may be combined to produce correlation matrices before being further processed.

Stress analysis, as stated earlier, includes features associated with a single subject. Standard feature analysis may include, but are not limited to: a) coordinated movements of (x,y) facial nodes to yield facial expressions such as (smiling, frowning, etc.) that can be further classified as emotions (such as anger, disgust, fear, happiness, sadness, surprise, and neutral). Typically negative valence emotions (ex. fear) map to feelings of stress; b) coordinated movements of (x,y) body nodes to yield expressions such as high frequency of movement (fidgeting), repetitive cyclical patterns (self-soothing behaviors); c) vocal features such as increases in the fundamental frequency and increases in harmonics-to-noise ratio; and d) linguistic feature analysis (ex. natural language processing for sentiment analysis).

Stress classifications can be obtained via standard coding libraries or through standard machine learning techniques. Any form of machine learning known in the art may be employed for this purpose, such as supervised machine learning, unsupervised machine learning, reinforcement machine learning, semi-supervised machine learning, or a hybrid form of machine learning that incorporates one or more of these different types of machine learning algorithms.

Stress can decrease the ability or one or more people to pay attention to another person, and subsequently decrease their ability to form strong connections. Stress measurements may be reported independently on the user dashboard. This reporting will notify the user of potentially harmful interactions or environmental settings, which will allow the user to take action (ex. remove person/people from the interaction, file disciplinary reports, provide training or counseling, etc.). To improve the accuracy of the rapport measurements, the stress analysis may be incorporated as features into the report analysis.

Rapport analysis, as stated earlier, compares features across different individuals via the similarity metric described earlier. These features are given weights of importance by the machine learning classifier. Any form of machine learning known in the art may be employed for this purpose, such as supervised machine learning, unsupervised machine learning, reinforcement machine learning, semi-supervised machine learning, or a hybrid form of machine learning that incorporates one or more of these different types of machine learning algorithms. The output results are presented to the user as graphs, charts, tables and text descriptions.

Furthermore, classification may be applied to each feature independently, or as an ensemble classification. Ensemble classification can be performed by a number of methods, such as for example a voting-based (majority vote) or as an average and can occur deep within the classification process or at the end. Classification may also be applied to a normalized feature cohort.

The user interface includes a video of the interaction with a graphical display either underneath the video or as an overlay at the bottom of the video. The user is able to customize or switch the view of the analysis. Possible views include timeline graphs of the rapport analysis, stress analysis for one or more people, and/or subsets of features used in the analysis.

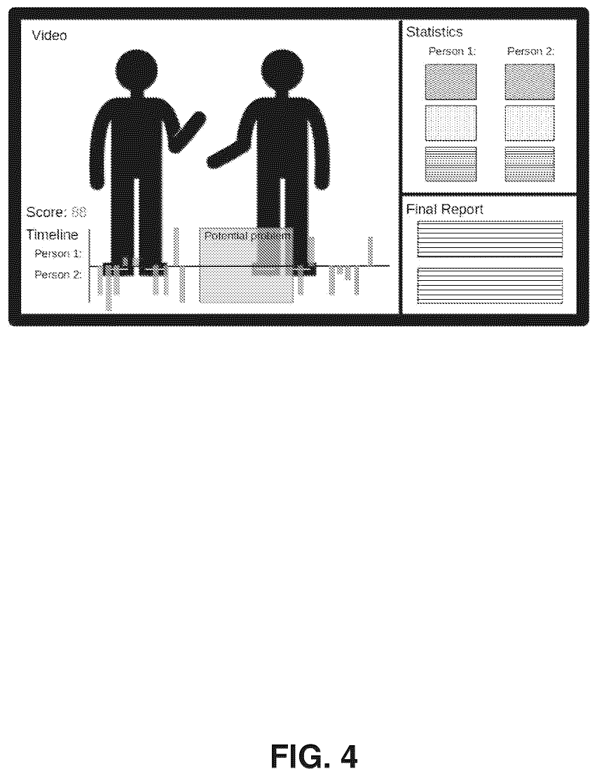

In FIG. 4, a two-person interaction is analyzed and displayed in a user interface (UI) based on analysis of each subject's audio output and physical gestures/movement output. The system generates a score based on these outputs and a negative interaction is detected when the measured outputs enter a problem zone as illustrated by the rectangular area. More specifically, large time segments of uncorrelated behavior between the interacting people as measured by audio and/or physical movement outputs and/or heightened stress levels reflected in the audio and/or physical gesture outputs appear in the problem zone. However, other indicators may be used to show these negative interactions, such as an oscillator that moves from a positive interaction zone to a negative interaction zone. Other tools may be used to represent a continuum of interactions that fluctuate between positive interactions and negative interactions such as graphs, bar charts, histograms, etc. In addition, colors such as green may be used to represent positive interactions and red to represent negative interactions. One of ordinary skill in the art will appreciate that these are just examples of many possible ways of representing positive and negative interactions in a UI.

The graphical display contains a timestamp that references back to the audio-visual stream. When the user clicks on the timeline graph, the audio-visual stream synchronizes to that moment in the conversation for easy playback, and additional analysis details (graphs, charts, descriptive text) pertaining to that timestamp are provided in a side panel. This one click feature allows users to easily access all the analysis information pertaining to that moment in time in the conversation. The user can customize or change what data they would like displayed.

The analysis output can be exported in part or in full by the user. The export format can be in any standard file, image, or database format such as ASCII, Portable Document Format (pdf), Rich Text Format, Joint Photographic Experts Group (jpg), Comma Separated Values (csv). Additionally, the output could be exported in a format that is easily transferrable and integrable into Human Resource Software systems, Learning Management Systems, Electronic Health Records or other database platforms that users commonly use for managing information about employees, students, patients, etc.

As discussed above, other audio and/or visual outputs from the subjects may be analyzed to evaluate the degree of mimicked behavior. A certain degree of mimicking or amount of mimicking behavior within a predefined range may indicate positive interactions, while mimicked conduct that falls outside of the range (i.e., too much or too little mimicking behavior) may indicate negative interactions. Accordingly, audio and/or physical gestures/movement output of each of the subjects may be analyzed to determine the amount of mimicked behavior taking place over a period of time. This analysis may be performed in conjunction with or separate from the stress analysis to evaluate positive and negative interactions between two or more subjects. The term “mimicking” is not intended to be strictly interpreted as copying such as mocking or comic mimicking, but rather is intended to cover generally imitative behavior that reflects a subconscious form of validation or admiration. Therefore, audio and physical gesture outputs are analyzed with this form of mimicking in mind. The subject's respective audio and physical gesture/movement outputs may be analyzed either separately or in combination to determine positive or negative interactions.

Alternatively, the system of the present invention may employ a form of machine learning to recognize correlations of physical gestures/movements between interacting people as an expression of positive or negative interaction. Similarly, correlations of certain words, phrases, sentences, tones, volumes, and inflections, either alone or in combination, may be recognized as an expression of positive or negative interaction. More specifically, the system may employ a machine learning algorithm to generate improved operations for correctly recognizing positive or negative interactions. Any form of machine learning known in the art may be employed for this purpose, such as supervised machine learning, unsupervised machine learning, reinforcement machine learning, semi-supervised machine learning, or a hybrid form of machine learning that incorporates one or more of these different types of machine learning algorithms.

According to an embodiment of the invention a UI dashboard is provided that includes an audio-video feed displayed on the dashboard. An overlay on the video may include one or more of the following: the current interaction score; an averaged classification over a discrete time window segments (e.g., 1 minute) color coded, or as speedometer; a timeline in sync with the audio-video segment, so that what is happening in the video corresponds with measurements in the timeline; rapport and stress measurements as a function of time; areas of interest automatically flagged; problems flagged; a side panel that can be hidden or viewed with statistics and feature category breakdown which allows the user to identify specific behaviors that contribute to communication problems; a selection of relevant raw features or independently classified features; and stress measurements, rapport measurements, or both.

The system may also generate: a final report, including a global score generated from an averaged rapport measurement over an extended period of time (e.g., 15 minutes); list of events of interest in the form of links, where a user can click on an event to migrate to that time in the video and timeline for easy viewing; an explanation of scores; and recommendations.

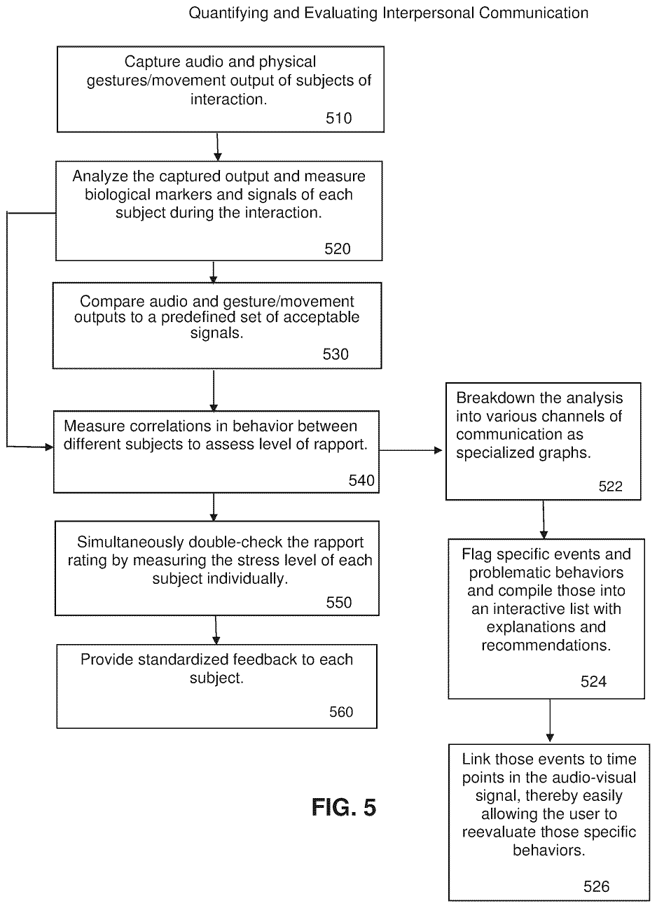

According to another embodiment illustrated in FIG. 5 a method or process of quantifying and evaluating interpersonal communication is disclosed. At step 510 a camera captures audio and physical gestures/movement output of two or more subjects of an interaction. At step 520, the captured output is analyzed, and biological markers and signals of each subject are measured. The biological markers may include physical movements, gestures, gesticulations, mannerisms, facial expressions, eye movement and the like as well as changes in body temperature, perspiration, heart rate, blood pressure, and pupil dilation. Signals may include speech patterns, pace of speech, volume, words, phrases, sentences, tone, inflection, and the like. At step 530 the audio and gesture/movement outputs are compared to a predefined set of acceptable markers or signals. Acceptable markers may include any recognized or known gestures/patterns of movement associated with positive interactions, as well as physiological markers (e.g., temperature, blood pressure, heart rate, etc.) within predefined “normal” parameters. Acceptable signals may similarly include recognized or known words, phrases, sentences, tones, inflections, speech patterns, speech pace, volume ranges associated with positive interactions. One of ordinary skill in the art will appreciate that “positive interactions” may be defined by a range of behaviors or speech on a continuum from extremely positive interactions to extremely negative interactions. In addition, audio output of a subject is primarily directed to speech, but may also include other sounds such as coughing, throat clearing, stuttering, and interjections (e.g., mmm, umm, mm-hmm, etc.).

At step 540, correlations in behavior between different subjects are measured to assess level of rapport. Correlations refer to a relatively high degree of similarity or mimicking and may be defined by a range on a continuum that goes from extremely disparate behavior to extremely similar behavior. A rapport rating may then be assigned to the interaction based on the measured correlations. At step 550, the system simultaneously double-checks the rapport rating by measuring the stress level of each subject individually. Stress level may be indicated by physiological markers such as body temperature, blood pressure, heart rate, and perspiration, physical gestures or audio output. Machine learning algorithms may be employed to improve the rate and accuracy of recognizing changes in stress levels. At step 560, the system provides standardized feedback concerning their interaction to each subject.

The analysis performed at step 540 can be further divided into steps 522-526. Specifically, at step 522, the analysis may be broken down into various channels of communication as specialized graphs. For example, one or more biological/physiological markers and signals can be defined as a feature, and one or more features may define a channel of communication that graphically oscillates based on the dynamic characteristics of the feature during the interaction.

At step 524, the system flags specific events and problematic behaviors and compiles those into an interactive list with explanations and recommendations. An event may be defined as a series of interactive behaviors such as speech and physical movements/gestures and problematic behaviors may include interactive behaviors or events that result in higher stress levels or are classified as problematic based on one or more measured features. The system can then generate an explanation of why the event or problematic behavior was flagged and how to reduce or avoid such events or behaviors in future interactions.

At step 526, the system links the flagged events or behaviors to time points or clips in the audio-visual signal, thereby easily allowing the user to review those specific behaviors in the clips. One of ordinary skill in the art will appreciate that the method steps discussed above may be performed in any order, in a specific order, or performed simultaneously.

Traditionally, a computer program or algorithm consists of a finite sequence of computational instructions or program instructions. It will be appreciated that a programmable apparatus (i.e., computing device) can receive such a computer program and, by processing the computational instructions thereof, produce a further technical effect.

A programmable apparatus includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

In view of the foregoing, it will now be appreciated that elements of the block diagrams and flowchart illustrations support combinations of means for performing the specified functions, combinations of steps for performing the specified functions, program instruction means for performing the specified functions, and so on.

It will be appreciated that computer program instructions may include computer executable code. A variety of languages for expressing computer program instructions are possible, including without limitation C, C++, Java, JavaScript, assembly language, Lisp, and so on. Such languages may include assembly languages, hardware description languages, database programming languages, functional programming languages, imperative programming languages, and so on. In some embodiments, computer program instructions can be stored, compiled, or interpreted to run on a computer, a programmable data processing apparatus, a heterogeneous combination of processors or processor architectures, and so on.

In some embodiments, a computer enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computer can process these threads based on priority or any other order based on instructions provided in the program code.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

The functions, systems and methods herein described could be utilized and presented in a multitude of languages. Individual systems may be presented in one or more languages and the language may be changed with ease at any point in the process or methods described above. One of ordinary skill in the art would appreciate that there are numerous languages the system could be provided in, and embodiments of the present invention are contemplated for use with any language.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

1. A computer-implemented method for segmenting and classifying multimodal data based on feature extraction and calculated distance metrics of the behavior of two or more individuals depicted in the multimodal data, the method executed by one or more processors and comprising:
(a) capturing a first set of multimodal data, said first set of multimodal data comprising (i) audio data and (ii) imaging data, wherein the imaging data includes movement data;
(b) segmenting the first set of multimodal data into a first subject multimodal dataset and a second subject multimodal data set;
(c) registering, for each of the first subject multimodal dataset and second subject multimodal data set: (i) individual-specific coordinate frames, and (ii) denoising using a trained filter;
(d) generating, movement outputs for a first individual in the first subject multimodal dataset, based at least in part on the individual-specific coordinate frames registered in the first subject multimodal dataset;
(e) generating, movement outputs for a second individual in the second subject multimodal dataset, based at least in part on the individual-specific coordinate frames registered in the second subject multimodal dataset;
(e) analyze movement outputs of the first individual and the second individual to generate a coordinated movement output dataset;
(f) calculating similarity metrics in the coordinated movement output dataset to assess common behavioral characteristics between first individual and second individual;
(g) identifying problematic behaviors in said similarity metrics in the coordinated movement output dataset and generating a problematic behavior dataset;
(h) linking events in said problematic behavior dataset to time points in multimodal data; and
(i) outputting the problematic behavior dataset as linked to the multimodal data to a graphical user interface.

2. The computer implemented method of claim 1, wherein the imaging data comprises at least one imaging modality selected from optical imaging, digital imaging, analog imaging, ultrasound, IR imaging, and LiDAR imaging.

3. The computer implemented method of claim 1, wherein capturing of the multimodal data is conducted via one or more sensors selected from the group comprising a motion sensor, an IR sensor, an ultrasound sensor, a digital imaging sensor, an analog imaging sensor, an audio sensor, a microphone, a physiological sensor, a vital sensor, an implantable sensor, a wearable sensor, and a LiDAR sensor.

4. The computer implemented method of claim 1, wherein the multimodal data further comprises stress data.

5. The computer implemented method of claim 4, wherein the stress data comprises bioinformatic information related to the first and second individual, wherein the bioinformatic information comprises one or more of: i) heart rate data; ii) respiration data; iii) perspiration data; iv) pupil dilation data; and v) micromovement data.

6. The computer implemented method of claim 5, further comprising the steps of:
analyzing the stress data for the first individual to create a first individual stress level dataset;
analyzing the stress data for the second individual to create a second individual stress level dataset;
analyzing the problematic behavior dataset, the first individual stress level dataset and second individual stress level dataset to generate a rapport rating; and
outputting, via said graphical user interface, standardized feedback related to said rapport rating.

7. The computer implemented method of claim 1, wherein calculating similarity metrics and identifying problematic behaviors in said similarity metrics comprises applying a convolutional neural network-based segmentation model.

8. The computer implemented method of claim 7, wherein convolutional neural network-based segmentation model is trained on an annotated behavioral analysis structure.

9. A non-transitory computer readable medium storing instructions that, when executed by a processor, cause the processor to perform a method for segmenting and classifying multimodal data based on feature extraction and calculated distance metrics of the behavior of two or more individuals depicted in the multimodal data, the method executed by one or more processors and comprising:
(a) capturing a first set of multimodal data, said first set of multimodal data comprising (i) audio data and (ii) imaging data, wherein the imaging data includes movement data;
(b) segmenting the first set of multimodal data into a first subject multimodal dataset and a second subject multimodal data set;
(c) registering, for each of the first subject multimodal dataset and second subject multimodal data set: (i) individual-specific coordinate frames, and (ii) denoising using a trained filter;
(d) generating, movement outputs for a first individual in the first subject multimodal dataset, based at least in part on the individual-specific coordinate frames registered in the first subject multimodal dataset;
(e) generating, movement outputs for a second individual in the second subject multimodal dataset, based at least in part on the individual-specific coordinate frames registered in the second subject multimodal dataset;
(f) analyze movement outputs of the first individual and the second individual to generate a coordinated movement output dataset;
(g) calculating similarity metrics in the coordinated movement output dataset to assess common behavioral characteristics between first individual and second individual;
(h) identifying problematic behaviors in said similarity metrics in the coordinated movement output dataset and generating a problematic behavior dataset;
(i) linking events in said problematic behavior dataset to time points in multimodal data; and
(j) outputting the problematic behavior dataset as linked to the multimodal data to a graphical user interface.

10. The non-transitory computer readable medium of claim 9, wherein the imaging data comprises at least one imaging modality selected from optical imaging, digital imaging, analog imaging, ultrasound, IR imaging, and LiDAR imaging.

11. The non-transitory computer readable medium of claim 9, wherein capturing of the multimodal data is conducted via one or more sensors selected from the group comprising a motion sensor, an IR sensor, an ultrasound sensor, a digital imaging sensor, an analog imaging sensor, an audio sensor, a microphone, a physiological sensor, a vital sensor, an implantable sensor, a wearable sensor, and a LiDAR sensor.

12. The non-transitory computer readable medium of claim 9, wherein the multimodal data further comprises stress data.

13. The non-transitory computer readable medium of claim 12, wherein the stress data comprises bioinformatic information related to the first and second individual, wherein the bioinformatic information comprises one or more of: i) heart rate data; ii) respiration data; iii) perspiration data; iv) pupil dilation data; and v) micromovement data.

14. The non-transitory computer readable medium of claim 13, further comprising the steps of:
analyzing the stress data for the first individual to create a first individual stress level dataset;
analyzing the stress data for the second individual to create a second individual stress level dataset;
analyzing the problematic behavior dataset, the first individual stress level dataset and second individual stress level dataset to generate a rapport rating; and
outputting, via said graphical user interface, standardized feedback related to said rapport rating.

15. The non-transitory computer readable medium of claim 9, wherein calculating similarity metrics and identifying problematic behaviors in said similarity metrics comprises applying a convolutional neural network-based segmentation model.

16. The non-transitory computer readable medium of claim 15, wherein convolutional neural network-based segmentation model is trained on an annotated behavioral analysis structure.

17. A system comprising: a processor, a memory storing instructions, and a display, wherein the processor is configured to execute the instructions to perform a method segment and classify multimodal data based on feature extraction and calculated distance metrics of the behavior of two or more individuals depicted in the multimodal data, the method comprising:
(a) capturing a first set of multimodal data, said first set of multimodal data comprising (i) audio data and (ii) imaging data, wherein the imaging data includes movement data;
(b) segmenting the first set of multimodal data into a first subject multimodal dataset and a second subject multimodal data set;
(c) registering, for each of the first subject multimodal dataset and second subject multimodal data set: (i) individual-specific coordinate frames, and (ii) denoising using a trained filter;
(d) generating, movement outputs for a first individual in the first subject multimodal dataset, based at least in part on the individual-specific coordinate frames registered in the first subject multimodal dataset;
(e) generating, movement outputs for a second individual in the second subject multimodal dataset, based at least in part on the individual-specific coordinate frames registered in the second subject multimodal dataset;
(f) analyze movement outputs of the first individual and the second individual to generate a coordinated movement output dataset;
(g) calculating similarity metrics in the coordinated movement output dataset to assess common behavioral characteristics between first individual and second individual;
(h) identifying problematic behaviors in said similarity metrics in the coordinated movement output dataset and generating a problematic behavior dataset;
(i) linking events in said problematic behavior dataset to time points in multimodal data; and
(j) outputting the problematic behavior dataset as linked to the multimodal data to a graphical user interface.

18. The system of claim 17, wherein the imaging data comprises at least one imaging modality selected from optical imaging, digital imaging, analog imaging, ultrasound, IR imaging, and LiDAR imaging.

19. The system of claim 17, wherein capturing of the multimodal data is conducted via one or more sensors selected from the group comprising a motion sensor, an IR sensor, an ultrasound sensor, a digital imaging sensor, an analog imaging sensor, an audio sensor, a microphone, a physiological sensor, a vital sensor, an implantable sensor, a wearable sensor, and a LiDAR sensor.

20. The system of claim 17, wherein the multimodal data further comprises stress data.
